(19) United States
(12) Patent Application Publication
(10) Pub. No.: US 2025/0255790 A1
(43) Pub. Date: Aug. 14, 2025

(54) METHOD FOR PRODUCTION OF A NAIL COATING HAVING GLASS MICROSPHERES

(71) Applicant: Wella Operations US LLC, Calabasas, CA (US)

(72) Inventors: Sunan Yuvavanich, Thousand Oaks, CA (US); Paul Harold Bryson, Hidden Hills, CA (US)

(21) Appl. No.: 19/040,216

(22) Filed: Jan. 29, 2025

Related U.S. Application Data
(60) Provisional application No. 63/553,247, filed on Feb. 14, 2024.

(51) Int. Cl. A61K 8/25; A61Q 3/02

(52) U.S. Cl. CPC A61K 8/25; A61Q 3/02; A61K 2800/412

(57) ABSTRACT

The present invention is directed to a method for preparing a solid coating with embedded glass microspheres on a fingernail plate and the coating produced therefrom.

### CLAIM OF PRIORITY

This patent application claims the benefit of priority to U.S. Provisional Application Ser. No. 63/553,247, filed Feb. 14, 2024, which is incorporated by reference herein in its entirety.

### FIELD OF INVENTION

The present invention is directed to a method for developing abrasion resistance for nail coating compositions. Microsphere glass beads are incorporated into the coating compositions as they are applied to natural nails. The microsphere glass beads do not dissolve, flatten or otherwise distort in the coating composition.

### BACKGROUND

Nail coating compositions have engendered an ageless reputation of beauty and elegance. Ancient Chinese dynasties demonstrated beauty through intricate dance with complex hand movements accentuated by nail coatings and extensions. With the advent of the late nineteenth and early twentieth centuries, nail coatings developed a cashet for enhanced beauty and glamor through their riveting brilliance and intricate design. Although some social circles of the time took a de-minimus view of artistic design for nail coatings, they nevertheless continued to care for nails and associated tissues by promoting health benefits. Today's societies embrace not only dazzling colors for lacquer coatings but also champion designs involving multi-colored intricate arrangements and nail extensions such as Ombre, Floral and Dot nails as well as French tips.

This said, today's societies also take a practical view toward the cosmetic and chemical underpinnings of nail coatings. Not only should such beauty and glamor aspects be appropriate and impressive for the particular societal circumstance of the day but they should also be environmentally and medically safe, enable reasonable wear and care properties and involve personal choice.

For nail coatings, wear and care properties have traditionally been enhanced by incorporation of solid polymer particles. The polymer particles such as poly(ethyl methacrylate) (PEMA) or poly(methyl methacrylate) (PMMA) deliver to the nail coatings additional abrasion resistance due to their particulate hardness and extremely dense cross-linked polymer network. Incorporation of polymer particles into a lacquer coating or a curable gel coating also involves at least partial softening and/or dissolution of the polymer network at the surfaces of the polymer particles. This phenomenon enables production of a smooth coating surface that is free of surface undulations, irregularities and bumps that would otherwise result from the presence of hard particles on and in the surface of the coating. This phenomenon also produces tendrils or threads of particle polymer extending from the particles themselves. These threads contribute to the strength of the coating by their non-covalent entwining with the lacquer and/or curable gel polymers.

Nevertheless, the nail coatings with incorporated polymer particles do not exhibit the abrasion resistance expected based upon the hardness of the particles themselves. While this lower abrasion resistance may be a result of the particle softening, the overall resistance of the coating should be enhanced by the incorporation of the particle polymer tendrils throughout the coating material. Attempts have been made to alter the abrasion resistance of such coatings by changing the chemical composition of the polymer particles, by changing the lacquer or gel composition and/or by altering the interaction between the particle tendrils and the lacquer or gel composition. However, the abrasion resistance remains an issue to be solved.

Therefore, development of abrasion resistance, hardness and flexibility for nail coatings constitutes at least some of the design parameters for modern nail coatings.

### SUMMARY OF THE INVENTION

These and other needs are achieved by the present invention which constitutes incorporation of glass bead microspheres into a polymerizable monomer and/or preformed film former for a nail coating. While glass microspheres according to the invention do not provide tendrils extending from the microsphere surfaces to entwine with the components of the nail coating composition, it has been found that glass microspheres having an average diameter of from about 25 microns (μ) to about 55 microns (μ), preferably about 30 μ to about 50 μ, more preferably about 35 μ to about 45 μ, wherein the deviation of each micron number is ±5 μ, deliver a smooth nail coating surface for such coating compositions and are close-packed to the extent that significant abrasion resistance is provided. In particular, these aspects of the present invention are directed to a method for preparing a particulate solid nail coating on one or more nail plates, a kit for practice of the method and a particulate solid nail coating on one or more nail plates exhibiting superior abrasion resistance.

The aspect of the invention directed to the method concerns applying to a nail plate such as a natural nail or artificial nail on a finger or a natural nail on a toe an adherent liquid to form an adherent nail coating followed by contacting the adherent nail coating with a portion of glass microspheres having an average spheric diameter of from about 25 μ to about 55 μ to adhere at least some glass microsphere onto the adherent nail coating and form a composite nail coating. Optionally, the step involving application of the adherent liquid and glass microspheres may be repeated at least once and preferably at least two or three times to prepare the composite nail coating. Following formation of the composite nail coating through one or more application steps as described, the composite nail coating may be treated to form the particulate solid nail coating which exhibits superior abrasion resistance. The treatment depends upon the chemical composition of the primary component of the adherent liquid. The treatment is designed to cure and/or convert the primary component of the adherent liquid into a hard solid coating.

According to these aspects of the invention, the adherent liquid may include a primary component chosen from any class of chemical composition that will enable glass microsphere adherence and may be transformed into a hard solid coating by chemical interaction and/or by drying. The classes of chemical compositions that may function as the primary component of the adherent liquid include at least:
a) A cyano(meth)acrylate monomer;
b) A (meth)acrylate monomer;
c) A UV curable monomer/dimer composition;
d) A non-reactive preformed film former; or
e) Any combination of a)-d).

The adherent liquid may optionally include at least a non-reactive volatile organic solvent and if the adherent liquid is a cyano(metho)acrylate monomer, the non-reactive volatile organic solvent will be anhydrous.

According to an aspect of the invention, treatment to cure and/or convert the adherent liquid depends upon the chemical composition of the primary component of the adherent liquid. When the primary component is the cyano(meth)acrylate monomer, treatment is accomplished by contacting the applied cyano(meth)acrylate and adhered glass microspheres with water, a hydroxylic organic liquid, an amine accelerator or any combination thereof, optionally in combination with a non-reactive organic solvent. When the primary component is the (meth)acrylate monomer, treatment is accomplished by contacting the applied (meth)acrylate and adhered glass microspheres with a peroxide initiator and an amine accelerant optionally in a nonreactive volatile organic solvent. When the primary component is the UV curable monomer/dimer composition, treatment is accomplished by exposing the composition to UV light. When the primary component is the preformed film former, treatment is accomplished by air evaporation of non-reactive volatile organic solvent dissolving the film former.

Preferably, the primary component is the cyano(meth)acrylate monomer with a tertiary amine with optional water as the treating agent.

Upon polymerizing, curing, UV curing and/or drying, the resultant polymerized, cured, UV cured and/or dried primary component constitutes the solid nail coating in which is embedded the glass microspheres as particulates.

A further aspect of the invention is directed to incorporation of one or more optional ancillary agents into the adherent liquid. The optional ancillary agent may deliver color, thixotropic properties, thickening properties, leveling properties and flexibility properties to the adherent liquid in its liquid state and/or in its cured and/or polymerized and/or solidified state.

Another aspect of the invention is directed to a kit for practice of the method according to the invention. The kit includes but is not limited to individual, separate containers or units of the adherent liquid, the glass microspheres, the treatment agent and optional color selections of pigment as well as measuring and mixing container and associated brushes and stirrers.

A further aspect of the invention is directed to a particulate solid nail coating on a nail plate wherein the coating is embedded with glass microspheres having a spherical diameter of from about 25 microns (μ) to about 55 microns (μ) and the coating is a cured and/or solidified composition including a treated primary component selected from the group consisting of polymerized cyano(meth)acrylate, polymerized (meth)acrylate monomer, polymerized monomer/dimer cured with UV radiation, and preformed film forming polymer. The particulate nail coating preferably presents a smooth surface and is at least significantly abrasion resistant. The particulate nail coating is prepared according to the method outlined above through use of the kit outlined above.

### Definitions

Unless defined otherwise, all technical and scientific terms used herein have the same meaning as commonly understood by a person of ordinary skill in the art.

As used in the specification and the appended statements and claims, the singular forms “a,” “an” and “the” include plural referents unless the context clearly dictates otherwise. Also use of a plural term describing a thing or element includes the singular unless the context clearly dictates otherwise. For example, the term (meth)acrylates includes a single (meth)acrylate as well as multiples.

The term “may” in the context of this application means “is permitted to” or “is able to” and is a synonym for the terms “is” and “can.” The term “may” as used herein does not mean possibility or chance.

The term and/or in the context of this application means one or the other or both. For example, an aqueous solution of A and/or B means an aqueous solution of A alone, an aqueous solution of B alone and an aqueous solution of a combination of A and B. When more than two items are referred, the term and/or also means any combination of these multiple items as well as all and each.

The term “about” as used herein, when referring to a numerical value or range, allows for a degree of variability in the value or range, and is understood to mean +10%, of a stated value or of a stated limit of a range.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4. Similarly, values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range.

The terms “comprising”, “comprises” and “comprised of” as used herein are synonymous with “including”, “includes” or “containing”, “contains”, and are inclusive or open-ended and do not exclude additional, non-recited members, elements or method steps.

For the purposes of the presently claimed invention, ‘% by weight’ or ‘wt. %’ as used in the presently claimed invention is with respect to the total weight of the recited composition. Further, the sum of wt. % of all the compounds (components), as described herein, in the respective compositions add up to 100 wt. %. Accordingly, all percents of ingredients or components are given as weight-percentages relative to the total weight of the composition in which the ingredients and/or components are present, unless otherwise stated.

All average molecular weights of polymers are weight-average molecular weights, unless otherwise specified.

The term “substantially free” as the term is used herein means completely or almost completely; for example, a composition that is “substantially free” of a component either has none of the component or contains such a small amount that any relevant functional property of the composition is unaffected by the presence of the small amount of the component in question. A compound that is “substantially pure” has only negligible traces of impurities present.

The term “substantial” means a significant amount such as more than a majority amount. For example, a mixture of compounds A and B in which A is present in a substantial amount means that A is present at a weight percent or number of moles that is greater than the weight percent or number of moles of B. This term also means more than a minimal characteristic, examples of which include substantial flow or substantial treatment.

The following groups of terms are used throughout this application: 1) preferred, preferably and preferable; 2) more preferred, more preferably and more preferable, 3) especially more preferred, especially more preferably and especially more preferable; 4) most preferred, most preferably and most preferably; 5) especially most preferred, especially most preferably and especially most preferable; and 6) very especially most preferred, very especially most preferably and very especially most preferable. These groups convey a meaning of preference for a group of substituents, structures, moieties, components and compounds. The degree of preference is self-explanatory by the terms themselves. Within each group, the meanings of the synonyms, preferred, preferably and preferable are the same. There is no difference in meaning in the context of this application when a group is described in a particular sentence as preferred and then in another sentence this same group is described as preferably. Not all six categories of preference are used in this application to describe each and every substituent, formula, subgenus integer symbol and atom designator. In some instances, two or three categories are used while in other categories five or six categories are used. The degree of preference as expressed by these terms for members of series which progress from many to a few individually named components is self-explanatory and internally consistent for the particular series being described. Reference throughout this specification to “embodiment”, “one embodiment” or “preferred embodiment” or “more preferred embodiment” or “most preferred embodiment” means a feature of any one or more elements and can be used in connect with different elements of the invention.

The term film former means a fully formed polymer such as a poly(meth)acrylate, polyester, polyamide, polyurethane, cellulosic ether or ester or shellac that will form a contiguous film or layer when the film former in an organic or aqueous medium is coated on a substrate and dried to remove the medium. The film former typically does not undergo substantial cross-linking or other chemical reaction after its deposition as a film.

The term (meth)acrylate means either one alone and/or both of an acrylate ester and a methacrylate ester. When an alkyl methacrylate ester alone is to be described, the parenthesis around “meth” is excluded and the resulting term means the 1-methyl-1-carbanoyloxyalkyl ethene or alkyl methacrylate. In a similar fashion, the term alkyl acrylate means 1carbanoyloxyalkyl ethene or alkyl acrylate.

The term adherent in the context of adherent liquid means that the liquid has sufficient viscosity in a quiescent state to remain on a surface when a surface is coated with the liquid and the surface is positioned vertically or semi-vertically, e.g. at an approximate 45 degree angle with respect to vertical. The term adherent in this context also means that particles such as micron sized particles can be inserted into the liquid and do not thereafter undergo substantial movement in the liquid when the liquid is contacted by the particles. While the word adherent also implies adhesiveness, tackiness or stickiness, the adherent liquid can be but is not necessarily sticky or tacky of the sense that an adhesive glue is sticky or tacky.

The term surfactant means a zwitterionic, nonionic, anionic or cationic compound having lipophilic and hydrophilic qualities so that it can function to solubilize lipophilic substances in hydrophilic and/or aqueous media. A surfactant may also perform as a plasticizer in polymer compositions to provide flexibility to the polymer composition.

The term plasticizer means a compound the may be soluble to dispersible in a solid material such as a polymer and enables the macromolecular configuration of the mixture to exhibit flexibility not present in the polymer alone often by lowering the glass transition temperature curve of the polymer. Plasticizers for polyolefins and functionalized polyolefins are well-known. Examples include alkyl phthalate esters, alkyl adipate esters, alkyl sebacate esters, glycerol triacetate (triacetin) and acetyl tributyl citrate.

The term rheologic control agent means a rheology modifier that will thicken an otherwise free-flowing liquid composition. The modification renders the composition flowable when applied by spray, brush or other coating method but the composition will remain in a static position in a quiescent state. These agents are typically classed as thixotropic agents. Examples include polyvinyl alcohol, a polyethylene glycol, vegetable gum, laponites, and hydrocarbon wax.

The term alcohol means a mono, di, tri or polyol that can solubilize other components of the composition. Alcohols include methanol, ethanol, propanol, butanol, ethylene glycol (ethylene diol), glycerin (glycerol), propylene diol, or di-ethylene glycol, di-propylene glycol as well as glyme (methoxylated ethylene glycol). Preferred alcohols include but are not limited to methanol, ethanol, propanol, ethylene glycol, propylene glycol and glycerin.

The term gel means a liquid within a three-dimensional network that forms at least a semi-solid-like consistency in the static, quiescent state. The gel has sufficient rheologic control and/or density at rest (static state) to maintain continuous integrity of the liquid gel as a coating or layer on a flat or curved surface. The gel character of the liquid means that the liquid will not spontaneously flow off a surface on which it has been coated but can be readily removed by mechanical force such as by wiping with a cloth or tissue.

The term flowable means a liquid that will flow like water or an aqueous latex paint when contacted by mechanical means such as by a brush, sponge or other applicator.

Together, the terms gel and flowable mean that the liquid having these characteristics is thixotropic.

The term alkylenyl used in the present invention means a linear C2-C6 hydrocarbon chain with open valences at both termini. An example is hexylenyl of the formula —(CH₂)₆—.

The term nail plate means a human fingernail, a human toenail as well as an artificial nail extension. The term fingernail includes thumbnail, index fingernail, middle fingernail, ring fingernail and pinky fingernail.

The term “user” means the person preparing and applying the nail coating composition. The user may be, for example, a professional manicurist working in a salon and is different from the subject on whose nails the composition is to be applied. The user, for example, may also be identical to the person on whose nails the composition is applied.

In the following passages, different aspects of the subject matter are defined in more detail. Each aspect so defined may be combined with any other aspect or aspects unless clearly indicated to the contrary. In particular, any feature indicated as being preferred or advantageous may be combined with any other feature or features indicated as being preferred or advantageous. A particular feature, structure, or characteristic described in connection with an embodiment may not be the same as another feature, structure or characteristic of another embodiment of the presently claimed invention. Thus, appearances of the phrases “in one embodiment” or “an embodiment” or “in a preferred embodiment” in various places throughout this specification are not necessarily all referring to the same embodiment but may refer to different embodiments of the presently claimed invention. Furthermore, the features, structures, or characteristics in one or more embodiments may be combined in any suitable manner, as would be apparent to a person skilled in the art from this disclosure. Furthermore, while some embodiments described herein include some, but not other features included in other embodiments, combinations of features of different embodiments are meant to be within the scope of the subject matter, and form different embodiments, as would be understood by those skilled in the art. For example, in the appended claims, any of the claimed embodiments can be used in any combination.

Reference throughout this specification to “one embodiment” or “preferred embodiment” means that a particular feature, structure, or characteristic described in connection with the embodiment is included in at least one embodiment of the presently claimed invention. Thus, appearances of the phrases “in one embodiment” or “in a preferred embodiment” in various places throughout this specification are not necessarily all referring to the same embodiment but may refer to different embodiments of the presently claimed invention. Furthermore, the features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the subject matter, and form different embodiments, as would be understood by those in the art. For example, in the appended claims, any of the claimed embodiments can be used in any combination.

### DETAILED DESCRIPTION OF THE INVENTION

Aspects of the present invention deliver superior abrasion resistance to nail plate coatings through the embedment of glass microspheres having an average spherical diameter of from about 25 microns to about 55 microns, preferably about 30 μ to about 50 μ, more preferably about 35 μ to about 45 μ, wherein the deviation of each micron number is ±5 μ. The glass microspheres form a smooth coating surface and their tough, strong character at least in part provides the superior abrasion resistance. The glass microspheres may be embedded in a solid coating matrix of a cyano(meth)acrylate polymer, a (meth)acrylate polymer, a UV cured polymer of acrylic monomer and dimer, and/or a preformed film former.

### The Method

According to embodiments of the method of the invention, micron sized glass microspheres are embedded in the adherent liquid which is subsequently polymerized, cured or dried to form an abrasion resistant solid coating on a nail plate. The micron diameter range of the glass microspheres enables the microspheres to avoid clumping in the adherent liquid when applied and avoid an irregular bumpy surface when the adherent liquid with embedded microspheres is treated to form of the solid coating. It is believed that the micron diameter range provides substantially uniform close packing of the embedded microspheres and enables at least in part the abrasion resistance and significant strength of the solid coating.

The steps for formation of the abrasion resistant solid coating with embedded microspheres involve:
a) Applying the adherent liquid to the nail plate;
b) Contacting the applied adherent liquid with a portion of glass microspheres to form a composite nail coating;
c) Repeating steps a) and b) if needed;
d) Treating the composite nail coating to form the abrasion resistant solid coating.

The adherent liquid applied to the nail coat may have sufficient flowability to enable it to spread substantially evenly on the nail plate but have sufficient thickness to avoid run-off from the nail plate. These properties may be achieved by incorporation of thixotropic character for the adherent liquid or by slightly treating the adherent liquid immediately after application to solidify it at least slightly. In addition to the inherent adherent character of the adherent liquid, its partial solidification treatment may also help its ability to hold the glass microspheres.

Following application and optional partial treatment of the adherent liquid, the so coated nail plate is contacted with a portion of the glass microspheres. The contact may be accomplished by bringing together the coated nail plate and a portion of the glass microspheres. This contact may be practiced by dipping the coated nail plate into the portion of glass microspheres, or by spreading, sprinkling and/or dusting the glass microspheres onto the coated nail plate, or by spraying or dripping a dispersion of the glass microspheres in inert volatile organic solvent onto the coated nail plate. Typically, the contact may be practiced by moving the coated nail plate through a portion of the glass microspheres to embed microspheres in the adherent liquid. Excess glass microspheres that have not been embedded in the coating of adherent liquid may be removed by shaking the coated nail plate and/or brushing the coated nail plate.

The two step process of applying the adherent liquid to the nail plate, optionally at least partially solidifying the adherent liquid and contacting the coating of adherent liquid with glass microspheres may be repeated one or more times to provide additional coatings of adherent liquid and additional portions of embedded microspheres.

When sufficient glass microspheres have been embedded in the coating of adherent liquid to form a composite coating, the composite coating may be treated to polymerize, cure and/or solidify the adherent liquid of the composite coating and form the solid coating. The nature of the treatment step depends upon the chemical character of the primary component of the adherent liquid.

If the primary component is a cyano(meth)acrylate, the treatment step involves contact of the cyano(meth)acrylate with water and/or with a hydroxylic organic liquid and/or an amine accelerator or combination thereof. The water may be applied neat or preferably may be diluted with an organic solvent such as a C2-C4 alkyl monoalcohol.

If the primary component is a (meth)acrylate monomer, the treatment step involves contact of the (meth)acrylate monomer with a peroxide initiator and a secondary or tertiary alkyl amine accelerator. Preferably, the secondary or tertiary alkyl amine is combined with the (meth)acrylate monomer to form at least in part the adherent liquid before application of the adherent liquid to the nail plate. The peroxide initiator, such as benzoyl peroxide, may be combined with the adherent liquid as it is forming the composite coating to initiate the polymerization of the monomer. The peroxide initiator may be applied to or into the adherent liquid prior to or simultaneous with the application of the adherent liquid to the nail plate. Alternatively, the peroxide initiator may be combined with the portion of glass microspheres to be embedded in the adherent liquid. This combination may be accomplished by mixing an appropriate portion of the dry peroxide with the glass microspheres or by mixing an appropriate portion of the glass microspheres with a dispersion of the peroxide in inert volatile organic solvent.

If the primary component of the adherent liquid is a UV curable gel, it will be composed of a (meth)acrylate monomer, a bis (meth)acryloyloxy alkane or polyol oligomer or polyurethane oligomer as well as a photoinitiator. The treatment step involves UV radiation of the adherent liquid to photopolymerize and cure the UV curable gel.

If the primary component of the adherent liquid is a preformed film former, the treatment step involves evaporation of the organic or aqueous-organic medium in which the film former is dissolved. Preferably, the evaporation step is rapidly accomplished through the use of warm, forced air such as with an air dryer apparatus.

### Compositions

The adherent liquid composition incorporates as a primary component at least one of the four classes of chemical compositions that will form the solid coating when treated as described above. These four classes are:
a) The cyano(meth)acrylate monomer;
b) The (meth)acrylate monomer with free radical initiator;
c) The UV curable monomer/dimer composition; and
d) The non-reactive preformed film former.

### The Cyano(meth)acrylate Class

The cyano(meth)acrylate class comprises any one or more of the C2-C10 alkyl cyano(meth)acrylate esters including the ethyl, propyl, butyl, pentyl hexyl, septyl, octyl, nonyl and/or decyl esters. The well-known methyl cyanoacrylate ester is trademarked as “SuperGlue” and is rapidly polymerized in the presence of air because of the residual moisture in air. Although methyl cyanoacrylate ester may be formulated as the primary component of the adherent liquid composition, its polymerization in contact with air is so rapid that its use for an embedment of the glass microspheres may be in need of a hurried pace. Consequently, it is preferred to apply the higher alkyl cyano(meth)acrylates so that the rate of their polymerization in air is slow enough to enable a reasonable pace for glass microsphere embedment.

Accordingly, the alkyl cyano(meth)acrylate including the ethyl, butyl, hexyl, octyl and decyl esters are preferred as the class of chemical composition of the primary component of the adherent liquid. The rate of polymerization may also be controlled by use of the cyanomethacrylate ester instead of the cyanoacrylate ester. The methyl group substitution of the cyanoacrylate slows the corresponding rate of the polymerization. A balance of the rate of polymerization and the rate of development of an adherent character enables appropriate selection of the alkyl cyano(meth)acrylate for a reasonable pace of glass microsphere embedment. The balance may also depend upon the number of repetitions of the adherent liquid application and the application of the glass microspheres.

Preferably, application of this class of primary component of the adherent liquid may be performed with ethyl, propyl, butyl, hexyl and/or octyl cyanoacrylate and/or a mixture thereof, more preferably the ethyl, butyl and/or octyl cyanoacrylate and/or a mixture thereof, and especially more preferably the ethyl and/or octyl cyanoacrylate and/or a mixture thereof. A mixture of alkyl cyano(meth)acrylates may be appropriate in circumstances where a minor amount of rapid polymerization is desired followed by a slower rate of polymerization to form the solid coating. Such mixtures may include a minor weight percentage of a cyano(meth)acrylate ester having fast polymerization rate such as a methyl and/or ethyl cyanoacrylate and a major weight percentage of a cyano(meth)acylate ester having a slower polymerization rate such as a butyl or octyl cyanoacrylate or cyanomethacrylate. Weight percentages for the fast polymerization rate cyano(meth)acrylate ester may range from about 1 wt % to about 20 wt %, preferably from 1 wt % to about 25 wt %, relative to the total weight of the composition with the remainder weight percentage being a slower polymerization rate cyano(meth)acrylate ester. The total weight percentage of all cyano(meth)acrylate esters used for the primary component of this class may range from 90 wt % to 99 wt %, preferably 96 wt % to 99 wt %. Often but not always, no organic solvent will be present but some amount of the amine accelerant will make of the remaining wt percentage of the adherent liquid.

The adherent liquid with the primary component as the cyano(meth)acrylate class may also optionally include an anhydrous, non-hydroxyl, volatile organic solvent. Preferably, the concentration of the solvent is from about 0.5 wt % to about 10 wt %, more preferably from about 0.5 wt % to about 5 wt % with the remainder being the cyano(meth)acrylate ester itself. Examples of the solvent for this class of adherent liquid includes ethylene carbonate, propylene carbonate, chloroform, carbon tetrachloride, and/or a hydrocarbon such as hexane, heptane, octane. While esters and ketones such as ethyl acetate and/or methyl ethyl ketone may be incorporated as solvents for this class, they preferably are conditioned to be anhydrous and are combined with the cyano(meth)acrylate ester under very dry conditions. When the unit container with the adherent liquid primary component class of cyano(meth)acrylate and one or more of the above identified solvents is opened, moisture from the atmosphere will be introduced and will initiate polymerization. With a choice of cyano(meth)acrylate that has a less than extreme polymerization rate, this phenomenon will generate adhesiveness and/or increased adhesiveness for the adherent liquid while enabling glass microsphere embedment at a reasonable pace.

The treatment agent for the cyano(meth)acrylate class of primary component is water delivered as moisture, as a constituent of an organic solvent and preferably as a constituent of an amine accelerant diluted with an organic solvent. The amine accelerant may be a tertiary amine such as alkyl diphenyl amine, diphenyl amine or methyl dihexyl amine. The concentration of the amine accelerant and water may be in a range of from 0.1 wt % to 2 wt %, preferably 0.2 wt % to 1.5 wt % in organic solvent and the organic solvent concentration may be in a range of from about 2 wt % to about 10 wt % relative to the total weight of the adherent liquid.

Ancillary additives discussed below may also be included in minor amounts as long as the one or more ancillary additives do not react with the cyano(meth)acrylate class composition.

The cyano(meth)acrylate class is the preferred primary component with a tertiary amine such as alkyl diphenyl amine being a preferred amine accelerant.

Polymerization of this class of primary component in the adherent liquid occurs upon the contact of moisture/water and is hastened by the amine accelerant. The polymerized primary component constitutes the solid nail coating in which is embedded the glass microspheres as particulate.

### The (Meth)acrylate With Free Radical Initiator Class

The (meth)acrylate monomer class comprises one or more C1-C8 alkyl (meth)acrylate esters combined with a peroxide initiator, an amine accelerant, an optional inert, volatile organic solvent and an optional free radical scavenger.

Preferably, the alkyl (meth)acrylate may be a methyl, ethyl propyl, butyl, pentyl or hexyl (meth)acrylate with the acrylates preferred relative to the methacrylates. More preferably the alkyl(meth)acrylate may be a methyl, ethyl or propyl (meth)acrylate with the acrylates being preferred relative to the methacrylates. The (meth)acrylate class may also include a higher carbon count alkyl or cycloalkyl (meth)acrylate such as hexyl, cyclohexyl, decyl, isobornyl or norbornyl (meth)acrylate. The presence of such a higher carbon count alkyl (meth)acrylate may act as a reactive diluent to facilitate continued polymerization after the volatile organic solvent has dissipated. Mixtures of C1-C3 alkyl (meth)acrylate and C3-C8 alkyl (meth)acrylate with optional cycloalkyl (meth)acrylate may also constitute the (meth)acrylate monomer class. The lower alkyl (meth)acrylates may exhibit higher polymerization rates than the higher alkyl and cycloalkyl (meth)acrylates so that the resulting adherent liquid may rapidly show a desirable higher viscosity yet enable continued embedment of the glass microspheres. The concentration of the (meth)acrylate monomer as the primary component of the adherent liquid may range from about 70 wt % to about 99 wt %, preferably from about 80 wt % to about 99 wt % relative to the total weight of the adherent liquid.

Polymerization initiation for the (meth)acrylate class of the primary component of the adherent liquid is generated by the presence of a peroxide, preferably a solid peroxide such as dibenzoyl peroxide or dicumyl peroxide. While the peroxide is to be combined with the (meth)acrylate monomer and optional amine accelerant to initiate polymerization, it cannot be stored with the latter two components. Consequently, the peroxide may be maintained in a separate container. To initiate polymerization with the (meth)acrylate monomer, a portion of the solid peroxide may be mixed with the glass microspheres immediately prior to practice of the method embodiments of the invention. The solid peroxide may coat at least in part the glass microspheres and will initiate polymerization when a nail plate coated with the adherent liquid is combined with the glass microsphere-solid dibenzoyl peroxide mixture. Alternatively, a portion of the solid peroxide may be mixed with the adherent liquid immediately before application of the adherent liquid to the nail plate. The solid peroxide may be combined with the adherent liquid as a solid or may be premixed with an inert volatile organic solvent immediately before use. The concentration of peroxide to be used with the (meth)acrylate class of primary component of the adherent liquid may range from about 0.1 wt % to about 3 wt %, preferably from 0.1 wt % to about 2 wt %, more preferably about 0.1 wt % to about 1 wt % relative to the total weight of the composition containing the adherent liquid to be applied to the nail plate. The concentration of peroxide in optional inert volatile organic solvent may be back calculated to provide the foregoing concentration of peroxide.

The polymerization of the (meth)acrylate monomer is accelerated by the secondary or tertiary di or tri-C3-C6-alkyl or di or tri-aryl or alkaryl amine, preferably a tertiary amine, once polymerization is initiated by the peroxide. The amine accelerator may be combined with the (meth)acrylate monomer at any time before use and preferably is combined with the (meth)acrylate monomer in the unit container of the kit described below. Optionally, when the amine accelerator and (meth)acrylate monomer are provided as a mixture in a single container for the kit, an appropriate amount of a free radical scavenger may be included to prevent premature free radical polymerization of the monomer. The concentration of the amine accelerator may range from about 0.1 wt % to about 5 wt %, preferably from about 0.1 wt % to about 3 wt %.

The (meth)acrylate class of primary component of the adherent liquid may optionally include an inert organic solvent including but not limited to one or more C1-C6 monoalcohols, C2-C6 diols, C3-C6 ketones, C1-C3 alkyl acetates, chloroform, ethylene carbonate, propylene carbonate, C5-C10 hydrocarbons and any mixture thereof. Preferably, the organic solvent may be methanol, ethanol, propanol, diacetone alcohol, acetone, methyl ethyl ketone, methyl acetate, ethyl acetate, chloroform, ethylene carbonate, propylene carbonate, hexane heptane, octane and any combination thereof. The concentration of organic solvent optionally present in the (meth)acrylate class of adherent liquid may range from about 0.5 wt % to about 30 wt %, preferably from about 0.5 wt % to about 20 wt % relative to the total weight of the adherent liquid.

Curing of the (meth)acrylate class of primary component is initiated by the presence of the peroxide initiator. Warming the adherent liquid with this primary component and the presence of the amine accelerator will increase the rate of curing and produce the solid nail coating with embedded glass microspheres as particulates.

### The UV Monomer/Dimer Class

The UV monomer/dimer class of the primary component comprises a C1-C10 alkyl, preferably an C1-C3 alkyl (meth)acrylate ester monomer and a (meth)acrylate dimer such as but not limited to a C2-C6 alkylene di-terminated by a (meth)acryloyloxy group or a 2-22 unit ethylene oxide oligomer or 2-22 unit propylene oxide oligomer, a 2 to 22 unit 1,4-butylene oxide (tetrahydrofuran) oligomer or 2-22 unit alkyl or polyalkylene oxide urethane oligomer di-terminated by a (meth)acryloyloxy group. Preferably, the monomer may be any of the monomers listed for the (meth)acrylate/free radical initiator class, preferably a methyl and/or ethyl (meth)acrylate, more preferably methyl and/or ethyl acrylate. Preferably the dimer may be C6 alkylene diterminated by an acryloyloxy group or 1,5-ethylenyloxyethylenyl diacrylate or 1,7-ethylenyloxyethylenyloxyethylenyl diacrylate.

The monomer and dimer concentrations may be arranged so as to provide low to moderate to high cross linking, preferably high cross linking. A higher concentration of the dimer relative to the monomer will deliver higher crosslinking. A higher crosslinking result will contribute to a harder solid coating. The total concentration of monomer and dimer together may range from about 70 wt % to about 99 wt percent relative to the total weight of the adherent liquid, preferably from about 85 wt % to about 98 wt %, more preferably from about 90 wt % to about 98 wt %. The molar ratio of monomer to dimer wherein a mol of dimer with two unsaturations is counted as one mol may range from about 20:1 to about 2:1, preferably about 10:1 to about 3:1, more preferably about 10:1 to about 4:1.

A photoinitiator such as benzophenone, phosphine oxide, 1-hydroxycyclohexyl phenyl ketone, and similar type I and type II photoinitiators may be combined with the UV monomer/dimer class to enable photopolymerization. The concentration of photoinitiator may range from 0.01 wt % to about 2 wt %, preferably 0.01 wt % to about 1.5 wt % relative to the total weight of the adherent liquid.

The UV monomer/dimer class of the primary component of the adherent liquid may optionally include an inert organic solvent including but not limited to one or more C1-C6 monoalcohols, C2-C6 diols, C3-C6 ketones, C1-C3 alkyl acetates, chloroform, ethylene carbonate, propylene carbonate, C5-C10 hydrocarbons and any mixture thereof. Preferably, the organic solvent may be methanol, ethanol, propanol, diacetone alcohol, acetone, methyl ethyl ketone, methyl acetate, ethyl acetate, chloroform, ethylene carbonate, propylene carbonate, hexane heptane, octane and any combination thereof. The concentration of organic solvent optionally present in the (meth)acrylate class of adherent liquid may range from about 0.5 wt % to about 30 wt %, preferably from about 0.5 wt % to about 20 wt % relative to the total weight of the adherent liquid.

Photolysis with actinic radiation at wavelengths appropriate for the photoinitiator present converts the UV monomer/dimer class of primary component to the solid coating containing the glass microspheres as particulates.

### The Preformed Film Former

The preformed film former (PFF) class of the primary component comprises a preformed non-reactive film forming polymer such as nitrocellulose, cellulose acetate, cellulose acetate butyrate, tosylamide epoxy resin, acrylates copolymer, a polyester such as adipic acid/neopentylglycol/trimellitic anhydride copolymer and/or a stryrene/acrylates copolymer and any mixture thereof. Preferably, the PFF is one or more of nitrocellulose, cellulose acetate butyrate, tosylamide epoxy resin and/or an acrylates copolymer, more preferably a combination of nitrocellulose, polyester, tosylamide epoxy resin and an acrylates copolymer. The concentration of the PFF in the adherent liquid may range from about 70 wt % to about 95 wt %, preferably from about 70 wt % to about 90 wt %. The weight percent ratio of the PFF's of the more preferred PFF may range from 4:(2/2):1 to 1:(1/1):0.1 for nitrocellulose to (polyester and/or tosylamide epoxy resin) to acrylates copolymer.

The PFF class of primary component of the adherent liquid may optionally include an inert organic solvent including but not limited to one or more C1-C6 monoalcohols, C2-C6 diols, C3-C6 ketones, C1-C3 alkyl acetates, chloroform, ethylene carbonate, propylene carbonate, C5-C10 hydrocarbons and any mixture thereof. Preferably, the organic solvent may be methanol, ethanol, propanol, diacetone alcohol, acetone, methyl ethyl ketone, methyl acetate, ethyl acetate, chloroform, ethylene carbonate, propylene carbonate, hexane heptane, octane and any combination thereof. The concentration of organic solvent optionally present in the (meth)acrylate class of adherent liquid may range from about 0.5 wt % to about 30 wt %, preferably from about 0.5 wt % to about 20 wt % relative to the total weight of the adherent liquid.

Drying the adherent liquid containing the PPF class of primary component in appropriate volatile organic solvent produces the solid nail coating containing glass microspheres as particulates.

### Ancillary Agent

The adherent liquid may also optionally include one or more ancillary agents to provide rheology control, solubilization of individual components that are insoluble in each other, free radical scavenging, thixotropy, plasticization, adherent liquid spreading and/or color. Ancillary agents providing at least in part these features include but are not limited to soluble and/or particulate pigments, gums such as locust gum, guar gum, acacia gum and similar natural thickening agents, cationic polymers, mordenite and/or bentonite and/or hectorite clay, one or more surfactants as described above, one or more plasticizers as described above, a secondary preformed film former, a polysiloxane such as dimethicone or a polysilicone copolymer with ethylene oxide and/or propylene oxide oligomer or polymer block units, polyacrylic acid and/or acrylic acid/methylacrylate copolymer. The optional ancillary agent is conditioned with the proviso that it will be chemically reactive with the class of compound primarily constituting the adherent liquid.

### The Glass Microspheres

The glass microspheres are constructed of silicon dioxide glass such as soda lime silicate or borosilicate. Silica, silicate and silicon dioxide are synonyms for the amorphous SiO₂ polymer constituting the chemical constitution of the glass microspheres. The glass microspheres are commercially available from such sources as Cospheric LLC of Somis California. The glass microspheres have high sphericity, i.e., their deviation from sphericality is essentially null and they have close size tolerance.

The glass microspheres are substantially to essentially spherical in three-dimensional shape and are not irregularly shaped microparticles, crushed glass, glass micro-shards or non- spherical glass powder.

According to embodiments of the invention, the glass microspheres incorporated into the adherent liquid pursuant to the above-described method of the invention have an average spherical diameter variation of about +2 to 5 microns and have an average spherical diameter in the range of 25 microns to 55 microns, preferably about 30 μ to about 50 μ, more preferably about 35 μ to about 45 μ, wherein the deviation of each micron number is +5 μ. For example, glass microspheres having an average spherical diameter of 30 microns±4 microns or glass microspheres having an average spherical diameter of 40 microns±5 microns or glass microspheres having an average spherical diameter of 50 microns±5 microns may be used to form embodiments of the invention.

As the experimental section below delineates, glass microspheres of the same silica composition but having an average spherical diameter of less than 25 microns create lumps when combined with the adherent liquid. Glass microspheres of this lower average micron size do not orderly pack when contacted by and embedded in the adherent liquid. Instead, these lower micron sized glass microspheres clump or bunch together during this method. The result is the formation of a significant non-uniform distribution of these lower sized microspheres so as to produce the appearance of voids of no microspheres and clumps or bunched groups of microspheres distributed throughout the adherent liquid. A substantially uniform distribution of microspheres embedded in the adherent liquid is not produced when these lower average size glass microspheres are used. It is not understood why this phenomenon occurs at this lower average micron microsphere size. Therefore, glass microspheres of average spherical diameter less than 20 microns are excluded from embodiments of the present invention.

Similarly, as the experimental section below delineates, glass microspheres of the same silica composition but having an average spherical diameter of greater than 56 microns ±5 microns do not clump together to produce voids and bunched groups. Instead, these larger sized microspheres produce a surface texture that is rough with undulations, peaks and valleys. Although it is thought that the undulations may be due in part to the larger spherical diameters of these higher average size glass microspheres, the larger sized microspheres are nevertheless smaller in diameter than the cross-sectional depth of the adherent liquid in which the microspheres are embedded. Consequently, it is not understood why these larger spherical diameter size would lead to an unacceptable rough nail coating. Therefore, glass microspheres of average spherical diameter greater than 60 microns are excluded from embodiments of the present invention.

Embodiments of the invention utilize glass microspheres that are clear and are internally uncolored. These glass microspheres have smooth glass surfaces which are not coated with any organic, mineral or inorganic substance. Alternatively, the glass microspheres having internal coloring provided by organic and/or inorganic compounds that are known to color glass may be used as substitutes for the clear uncolored glass microspheres of embodiments of the invention. Exemplary internal colors of the glass microspheres may be provided by inclusion of cobalt oxide (blue-violet), manganese dioxide (purple), chromic oxide (green), cadmium sulfide (yellow), sulfur (amber), antimony (white), gold chloride (red), iron oxide (red to brown), selenium oxide (red shades), carbon oxide (amber-brown), copper compounds (red, green and blue). The concentration of element or mineral needed to produce the color is minor. Any possible toxicity from elements and minerals that have known toxic effects on humans is alleviated and eliminated by the encapsulation of the element or mineral in the glass.

### Pigment Color

The composite glass microsphere-adherent liquid and the resultant solidified coating may also be colored by incorporation of organic and/or inorganic pigment that is soluble and/or dispersible in inert, volatile organic solvent. The pigment in solvent may be combined with a portion of the glass microspheres to be embedded in the adherent liquid. The soluble pigment distributes on the surfaces of the glass microspheres and is carried by the microspheres to be co-embedded in the adherent liquid. Typically, the soluble pigment then redistributes throughout the adherent liquid before the adherent liquid is converted to a solid coating. The concentration of soluble pigment may range from 0.1 wt % to 5 wt % relative to the total weight of all components of the particulate nail coating. The concentration may depend upon the intensity hue and shade of the soluble pigment to be combined with the other components of the particular nail coating.

Examples of soluble pigment include but are not limited to pigment yellow 3, 14, 83, 65; pigment red 53:1, 122, 4; quinacridone, pigment orange 5; pigment blue 29; fast yellow; pigment violet 19 and other similar organic pigments.

### The Kit

The Kit embodiments according to the invention comprise separate units of the components for practicing embodiments of the method including components such as the adherent liquid, the glass microspheres, the agent or agents for treating, the optional pigment colors and the optional ancillary components described above. The Kit may be designed to provide sufficient amounts of the components to enable coating the nail plates of one or two hands. Alternatively, the Kit may be designed to provide separately bulk amounts of the components which may be measured to deliver appropriate amounts for use with the nail plates of one or two hands.

To practice the method of the invention, the Kit includes one or more measuring containers for determining appropriate amounts of the components of the adherent liquid for use with the nail plates of one or two hands, a bowl for containing an appropriate portion of the glass microspheres to enable contact between the glass microspheres and the adherent liquid coated on one or more nail plates, a container for holding and optionally mixing the measured amounts of primary component and other optional components such as but not limited to amine accelerant and peroxide initiator to form the adherent liquid and a container for holding the measured amount of agent or agents for treating. Additionally, the Kit may include a bottle of non-reactive organic solvent appropriate for the class of primary component of the adherent liquid chosen for practice of the method, an optional container for holding the measured amount of pigment color and an optional container for holding the measured amount or amounts of optional ancillary agents. One or more brushes for applying the adherent liquid and one or more stir rods for combining the adherent liquid and/or glass microspheres with other components may also be included.

The Kit may be packaged in a box or other appropriate packaging construct for holding the container units, measuring, mixing and holding containers, bottles, brushes and other materials needed for practice of the method according to the invention. Included in the Kit box may also be written instructions for measurement of the components for use on the nail plates of one or two hands, for mixing and application of the adherent liquid and for contact between the adherent liquid and the glass microspheres.

Typically, the instructions will explain that the contact between the adherent liquid and the glass microspheres may be accomplished by putting a nail plate coated with the adherent liquid into a portion of the glass microspheres. Other means of contact may be explained as a technique for spreading the glass microspheres onto the nail plate coated with adherent liquid. The spreading means may be accomplished through use of a provided sifter similar to an ordinary “salt-shaker” having perforations in its sifting top sized appropriately for passage of individual glass microspheres. The instructions may also explain a means of contact as a technique for combining the glass microspheres with a minor portion of appropriate non-reactive volatile organic solvent to provide a dispersion and dripping or spraying the dispersion onto the adherent liquid coated on the nail plate.

### The Particulate Solid Nail Coating

The particulate solid nail coating comprises the treated adherent liquid with embedded glass microspheres coated on a nail plate. The treated adherent liquid comprises a solid coating formed by polymerizing, curing and/or drying the class of primary component making up the adherent liquid. The resulting solid nail coating has high abrasion resistance and a smooth surface owing at least in part to the glass microspheres of the spherical diameter sizes of the micron range described above. The resulting solid nail coating may be clear or colored depending on whether pigment color is incorporated into the adherent liquid. The particulate solid nail coating exhibits excellent abrasion resistance.

### EXAMPLES

### Practice of the Method and Experimental Results

The traditional dip powder system is a nail enhancement system designed for long-lasting wear. Traditionally, the system includes a liquid part and a polymer powder part. In practice, the dip powder system uses a liquid resin as base coat and as a topcoat. The liquid resin is typically formulated with a very reactive monomer such as ethyl cyanoacrylate. The powder is made from an organic polymer of PEMA-Poly(ethyl methacrylate) and/or PMMA-Poly(methyl methacrylate). The solid organic polymer may be melt-extruded, hot melt sprayed or otherwise processed into very small beads having a typical diameter size in the range of 5 to 80 microns. The result is the PEMA and/or PMMA dip powder.

The traditional dip powder process involves brushing the liquid resin onto a nail plate and dipping the coated nail plate into a container holding the dip powder. At least some of the PEMA and/or PMMA micron sized beads adhere to the liquid resin. Removal of the powder/resin coated nail plate from the dip powder container and shaking away or brushing away non-adhered powder provides a first layer of resin and adhered dip powder. This process is typically repeated to produce multiple layers or stack on the nail plate. Color in the form of pigment may be added to the liquid resin to achieve a full coverage of color. The stack is then topped by an activator solution that accelerates curing of the cyanoacrylate resin.

As discussed above, PEMA and/or PMMA powders for dip coatings have associated issues relating to use, health, environment and abrasion resistance. Alternative substances such as ceramic and/or glass particles may come to mind. However, coatings with these kinds of hard, unmalleable particles are not expected to be fileable and shapable into smooth surfaces like coatings with PEMA/PMMA particles. It would be expected that such filing would dislodge the ceramic and/or glass particles leaving cratering in the coating. Moreover, the incorporation of ceramic and/or glass particles into a nail plate coating would be expected to render the coating inflexible unlike the flexible coatings made with PEMA/PMMA particles. The PEMA and/or PMMA particles partially dissolve into the liquid monomer and form a flexible tendril network throughout the cured resin. In contrast, ceramic and/or glass particles have no such properties. Moreover, the partial dissolution property of PEMA and/or PMMA particles renders the process insensitive to their particle size. A large variation of particle sizes is the norm because of this reason. Consequently, the typical wearer will be troubled by the expected inability to file and shape the resulting coating and will be troubled by the expected lack of coating flexibility typically afforded by PEMA and/or PMMA powders.

In view of the expected difficulties arising from the use of glass particles as a dip coating, it is surprising that a certain range of micron sizes of glass microspheres is able to deliver an abrasion resistant coating that maintains at least some flexibility, a smooth surface and minimizes the crater effect caused by filing.

In the following experiments, the optimum glass microsphere sizes for production of a desirable, fileable smooth coating (Experiment 1) and the workability of the resin-glass microsphere coating with colorant (Experiment 2) are assessed.

### Experiment 1

Glass Microsphere size

### Particulate Material

1. Commercial OPI Powder Perfection-Clear Setting PMMA/PEMA Powder Median Particle size 50-75 microns
2. Soda Lime Solid Glass Microspheres Median Particle size 8-12 microns
3. Soda Lime Solid Glass Microspheres Median Particle size 35-45 microns
4. Soda Lime Solid Glass Microspheres Median Particle size 67-87 microns
5. Barium Borosilicate Median Particle size 19 microns

### Resin Material

a) OPI Powder Perfection Base coat resin of ethyl cyanoacrylate 100 wt %
b) OPI Powder Perfection Top coat resin of ethyl cyanoacrylate 100 wt %
c) OPI Powder Perfection Activator tertiary amine in ethanol/water solution.

### Method

1. Weight approximately 20 grams of tested powder into small plastic cup. Record weight of a powder and a plastic cup.
2. Apply one coat of OPI Powder Perfection base coat to cover entire surface of nail spoon.
3. Immediately dip the same nail spoon at a 45 Degree angle into tested powder. Tap off excess powder back to plastic cup.
4. Use a cosmetic brush to wipe off any remaining loose powder.
5. Apply a second layer of OPI Powder Perfection Base Coat and tested Powder. Wipe any remaining loose powder.
6. Repeat step 2-4 for 2 times.
7. Apply one coat of OPI Powder Perfection Activator, making sure to cover the entire surface of the nail spoon. Allow it to dry completely.
8. Smooth the surface if needed with an OPI FLEX 220/280 Buffer. Use a cosmetic brush to remove excess powder and filing dust.
9. Apply another coat of OPI Powder Perfection Activator to the same nail spoon, making sure to cover the entire surface. Allow it to dry completely.
10. Apply one coat of OPI Powder Perfection Top Coat to each nail spoon and allow to dry. Record the time from application to touch dry.

### Evaluation of Properties of Powder Coatings Made With Powders 1-5

### Properties to Observe

Film appearance: visual perception on film clarity and color
Workability: ability to achieve smooth and even application
Powder weight per nail spoon (gram): the total weight of powder used per nail spoon calculated from final and initial weight of powder in plastic cup (after and before application).
Total coating weight per nail spoon (gram): the total weight of base coat, powder and top coat used per nail spoon calculated from final and initial weight of nail spoon (after and before application).
Film thickness (millimeter): measured by electronic digital caliper, the film thickness is calculated from final and initial film thickness of nail spoon (after and before application)
Touch dry (min): press thumb against the film. If the film is cured, the hardened layer should not feel tacky. Instead, it should feel dry to the touch.
Powder flowability: the rolling ability of powder over one another
Polish ability: capable of being filed or polished by nail file.

### Results

| | Material | | | | |
|---|---|---|---|---|---|
| | | Soda Lime | Soda Lime | Soda Lime | |
| | | Solid | Solid | Solid | |
| | OPI-Clear | Glass | Glass | Glass | Barium |
| | Powder | Micro- | Micro- | Micro- | Borosil- |
| | Perfection | spheres | spheres | spheres | icate |
| Ingredient | Poly(ethyl | Glass | Glass | Glass | Barium |
| | methac- | oxide | oxide | oxide | Borosil- |
| | rylate) | micro- | micro- | micro- | icate |
| | | spheres | spheres | spheres | |
| Particle | 50-75 | 8-12 | 35-45 | 67-87 | 19 |
| size (μm) | | | | | |
| Powder | opaque | opaque | opaque off | opaque off | opaque |
| Appearance | white | white | white | white | white |
| Film | slightly | slightly | slightly | opaque | opaque |
| Appearance | opaque | opaque | opaque | | |
| Workability | smooth, | create a | smooth, | rough | create a |
| | even | lump | even | surface | lump |
| | appli- | | appli- | | |
| | cation | | cation | | |
| Powder | 0.047 | 0.025 | 0.037 | 0.045 | 0.037 |
| weight | | | | | |
| per nail | | | | | |
| spoon (g) | | | | | |
| Coating | 0.26 | 0.12 | 0.22 | 0.24 | 0.22 |
| weight | | | | | |
| per nail | | | | | |
| spoon (g) | | | | | |
| Film | 0.590 | 0.373 | 0.438 | 0.546 | 0.540 |
| thickness | | | | | |
| (mm) | | | | | |
| Touch dry | 2:00-2:30 | 2:00-2:30 | 2:00-2:30 | 2:00-2:30 | 2:00-2:30 |
| (min) | | | | | |
| Powder | free | Not free | free | free | Not free |
| Flowability | flowing | flowing | flowing | flowing | flowing |
| Polish ability | yes | no | no | no | no |

### Experiment 2

Objective: Workability of optimum glass bead particle size with colorants

### Materials—Polymer Powder and Glass Microspheres

1. OPI Powder Perfection-Clear Setting Powder Median Particle size 50-75 microns
2. Soda Lime Solid Glass Microspheres Median Particle size 35-45 microns
   Materials—Resin, as described above
a) OPI Powder Perfection Base coat
b) OPI Powder Perfection Top coat
c) OPI Powder Perfection Activator

### Materials—Pigment Colors

1. Geopearl Soft Silver Particle size 1-15 microns
2. Geopearl C Crystal Silk Beige Particle size 5-25 microns
3. Geopearl C Skylight Silver Particle size 10-60 microns
4. Geopearl C Bright Morganite Particle size 10-100 microns

### Procedure for Method

1. Mix Soda Lime Solid Glass Microspheres with listed colorants using high speed mixer at 500 rpm for 30 seconds (formula below)

| Ingredients | Formula 1 | Formula 2 | Formula 3 | Formula 4 |
|---|---|---|---|---|
| Soda Lime Solid Glass | 98.0 | 98.0 | 98.0 | 98.0 |
| Microspheres | | | | |
| Geopearl Soft Silver | 2.0 | — | — | — |
| Geopearl C Crystal Silk | — | 2.0 | — | — |
| Beige | | | | |
| Geopearl C Skylight | — | — | 2.0 | 2.0 |
| Silver | | | | |
| Geopearl C Bright | — | — | — | — |
| Morganite | | | | |
| Total | 100.0 | 100.0 | 100.0 | 100.0 |

2. Weight approximately 20 grams of tested powder into small plastic cup. Record weight of a powder and a plastic cup.
3. Immediately dip the same nail spoon at a 45 Degree angle into tested powder. Tap off excess powder back to plastic cup.
4. Use a cosmetic brush to wipe off any remaining loose powder. Apply a second layer of OPI Powder Perfection Base Coat and tested Powder. Wipe any remaining loose powder.
5. Repeat step 2-4 for 2 times.
6. Apply one coat of OPI Powder Perfection Activator, making sure to cover the entire surface of the nail spoon. Allow it to dry completely.
7. Smooth the surface if needed with an OPI FLEX 220/280 Buffer. Use a cosmetic brush to remove excess powder and filing dust.
8. Apply another coat of OPI Powder Perfection Activator to the same nail spoon, making sure to cover the entire surface. Allow it to dry completely.
9. Apply one coat of OPI Powder Perfection Top Coat to each nail and allow to dry. Record the time from application to touch dry.
10. Evaluate properties below:
   Workability: ability to achieve smooth and even application
   Powder weight per nail spoon (gram): the total weight of powder used per nail spoon calculated from final and initial weight of powder in plastic cup (after and before application).
   Total coating weight per nail spoon (gram): the total weight of base coat, powder and top coat used per nail spoon calculated from final and initial weight of nail spoon (after and before application).
   thickness (millimeter): measured by electronic digital caliper, the film thickness is calculated from final and initial film thickness of nail spoon (after and before application)
   Touch dry (min): press thumb against the film. If the film is cured, the hardened layer should not feel tacky. Instead, it should feel dry to the touch.
   Powder Flowability: the rolling ability of powder over one another
   Polish ability: capable of being filed or polished by nail file.

### Results

| | Test Formula | | | | |
|---|---|---|---|---|---|
| | OPI | | | | |
| | Powder | | | | |
| | Perfection | | | | |
| | Rice Rice | Formula | Formula | Formula | Formula |
| | Baby | 1 | 2 | 3 | 4 |
| Colorant | N/A | 1-15 | 5-25 | 10-60 | 10-100 |
| Particle size, | | | | | |
| microns | | | | | |
| Workability | smooth, | smooth, | smooth, | smooth, | smooth, |
| | even | even | even | even | even |
| | appli- | appli- | appli- | appli- | appli- |
| | cation | cation | cation | cation | cation |
| Powder | 0.048 | 0.037 | 0.040 | 0.039 | 0.040 |
| weight per | | | | | |
| nail spoon | | | | | |
| (g) | | | | | |
| Coating | 0.24 | 0.22 | 0.23 | 0.24 | 0.24 |
| weight per | | | | | |
| nail spoon | | | | | |
| (g) | | | | | |
| Film | 0.56 | 0.40 | 0.42 | 0.44 | 0.41 |
| thickness | | | | | |
| (mm) | | | | | |
| Touch dry | 2:00-2:30 | 2:00-2:30 | 2:00-2:30 | 2:00-2:30 | 2:00-2:30 |
| (min) | | | | | |
| Powder | free | free | free | free | free |
| Flowability | flowing | flowing | flowing | flowing | flowing |
| Polish ability | yes | no | no | no | no |

The results of Experiments 1 and 2 demonstrate that a smooth cured resin coating with glass microspheres may be produced with microspheres ranging in micron size from 25 microns to 55 microns ±5 microns. This range is determined by the results of the optimum range of 35 to 45 microns and the downward taper of the optimum to clumping at 19 microns and bumpy surface at 65 microns. Accordingly, a smooth cured surface with the glass microspheres may be obtained at the range of 25 μ to 55 μ. However, the results show that polishing the coating surface after curing is not attainable. Consequently, the coating optimally may be shaped during an intermediate stage of curing. The presence of the close packed microspheres delivers a polished appearance without additional polishing with abrasive.

Alternatively, following the combination of the glass microspheres and the coating one or more times followed by curing to provide a finished cured coating with microspheres, the finished cured coating may be top coated with another portion of the adherent liquid and at least partially treated to cure, solidify and/or dry the adherent liquid. This topcoat would not include the glass microspheres. In its uncured or partially cured or dried state the topcoat can be shaped as desired to create any desired surface form. The uncured or partially cured topcoat with or without shaping may be cured, solidified and/or dried to a final topcoat. The final topcoat may be polished to enhance the shine of the surface. As explained above, the surface of the cured or solidified coating with glass microspheres and without such a topcoat already will exhibit a shiny, smooth surface.

### SUMMARY STATEMENTS

The inventions, examples and results described and claimed herein may have attributes and embodiments include, but not limited to, those set forth or described or referenced in this application.

All patents, publications, scientific articles, web sites and other documents and ministerial references or mentioned herein are indicative of the levels of skill of those skilled in the art to which the invention pertains, and each such referenced document and material is hereby incorporated by reference to the same extent as if it had been incorporated verbatim and set forth in its entirety herein. The right is reserved to physically incorporate into this specification any and all materials and information from any such patent, publication, scientific article, web site, electronically available information, textbook or other referenced material or document.

The written description of this patent application includes all claims. All claims including all original claims are hereby incorporated by reference in their entirety into the written description portion of the specification and the right is reserved to physically incorporated into the written description or any other portion of the application any and all such claims. Thus, for example, under no circumstances may the patent be interpreted as allegedly not providing a written description for a claim on the assertion that the precise wording of the claim is not set forth in haec verba in written description portion of the patent.

While the invention has been described in conjunction with the detailed description thereof, the foregoing description is intended to illustrate and not limit the scope of the invention, which is defined by the scope of the appended claims. Thus, from the foregoing, it will be appreciated that, although specific nonlimiting embodiments of the invention have been described herein for the purpose of illustration, various modifications may be made without deviating from the scope of the invention. Other aspects, advantages, and modifications are within the scope of the following claims and the present invention is not limited except as by the appended claims.

What is claimed is:

1. A method for preparing a particulate solid nail coating on a nail plate, comprising:
a) applying to a nail plate an adherent liquid to form an adherent nail coating thereon;
b) contacting the adherent nail coating with a portion of glass microspheres having average spheric diameter of from about 25 μ to about 55 μ to adhere at least some glass microspheres onto the adherent nail coating and form a composite nail coating;
c) Optionally repeating steps a and b at least once;
d) Treating the composite nail coating to form the particulate solid nail coating.

2. The method according to claim 1 wherein the adherent liquid comprises a primary component selected from the group consisting of:
a) at least a cyano(meth)acrylate monomer;
b) at least a (meth)acrylate monomer;
c) at least a UV curable composition of a monomer and dimer;
d) at least a non-reactive pre-formed film former.

3. The method according to any of the preceding claims wherein the adherent liquid has a viscosity that enables the adherent liquid to spread and cover essentially to completely the nail plate during its application and enables the adherent liquid to remain in place without spreading after its application.

4. The method according to any of the preceding claims wherein the adherent liquid is thixotropic.

5. The method according to any of the preceding claims wherein the treating step comprises treating the adherent liquid with an agent for solidification of the adherent liquid.

6. The method according to any of the preceding claims wherein the primary component of the adherent liquid is at least the cyano(meth)acrylate monomer and the agent is water, a hydroxylic organic liquid, an amine or any combination thereof optionally in the non-reactive volatile organic solvent.

7. The method according to any of the preceding claims wherein the primary component of the adherent liquid is at least the (meth)acrylate monomer and the agent is a secondary or tertiary amine accelerator and a peroxide initiator.

8. The method according to any of the preceding claims wherein the primary component of the adherent liquid is at least the UV curable combination of a (meth)acrylate monomer, a di-(meth)acrylate oligomer, a scavenger and a photoinitiator and the agent is UV radiation.

9. The method according to any of the preceding claims wherein the primary component of the adherent liquid is at least the preformed film former in non-reactive volatile organic solvent and the agent is air blower device for evaporation of the non-reactive volatile organic solvent.

10. The method according to any of the preceding claims wherein the adherent liquid optionally further comprises an ancillary agent that is non-reactive with the primary component and is selected from the groups consisting of one or more of a thickening agent, a leveling agent, a colorant, a mordenite clay, a preformed film forming polymer, a gum, a surfactant, a plasticizer, a polysiloxane a cationic polymer or any combination thereof.

11. The method according to any of the preceding claims wherein the preformed film forming polymer is selected from the group consisting of an olefinic polymer, a cellulosic polymer, an ester polymer, an amide polymer, a polyepoxide and any combination thereof.

12. The method according to any of the preceding claims wherein the non-reactive, volatile organic solvent is at least an alcohol, a ketone, a cyclic carbonate, an ester or a combination thereof and optionally is anhydrous.

13. The method according to any of the preceding claims wherein the glass microspheres are soda lime silicate glass, borosilicate glass or a combination thereof and are clear or colored.

14. The method according to any of the preceding claims further comprising applying a topcoat to the particulate solid nail coating.

15. The method according to any of the preceding claims wherein the glass microspheres have a preferable average spherical diameter of from about 30 μ to about 50 μ, and more preferably from about 35 μ to about 50 μ, wherein the deviation of the minimum and maximum of each range is ±5 μ.

16. The method according to any of the preceding claims wherein the adhered glass microspheres are substantially to essentially uniformly distributed in the particulate solid nail coating.

17. The method according to claim 16 wherein the particulate solid nail coating provides a smooth, wear resistant surface.

18. A kit for practice of the method of any of the preceding claims comprising in separate containers at least the glass microspheres in a first container, the adherent liquid in a second container and optionally the agent in a third container.

19. A particulate solid nail coating on a nail plate produced according to the method of any of claims 1-17.
